(19) United States
(12) Patent Application Publication
(10) Pub. No.: US 2025/0312391 A1
(43) Pub. Date: Oct. 9, 2025

(54) A Mixture of Probiotic Strains to Improve Health and Growth Performance of Ruminants

(71) Applicant: CHR. HANSEN A/S, Hoersholm (DK)

(72) Inventors: Jennifer SCHUTZ, Milwaukee, WI (US); Steven P. LERNER, Milwaukee, WI (US); Giuseppe COPANI, Parma (IT); Oscar QUEIROZ, Carrigtwohill (IE); Bruno Ieda CAPPELLOZZA, Hoersholm (DK)

(73) Assignee: CHR. HANSEN A/S, Hoersholm (DK)

(21) Appl. No.: 18/864,732

(22) PCT Filed: May 16, 2023
(86) PCT No.: PCT/EP2023/063076

§ 371 Date: Nov. 11, 2024

Related U.S. Application Data
(60) Provisional application No. 63/342,982, filed on May 17, 2022.

(51) Int. Cl. A61K 35/742; A23K 10/18; A23K 50/10; A61K 35/741; A61K 35/747; C12N 1/20; C12R 1/01; C12R 1/10; C12R 1/125; C12R 1/225

(52) U.S. Cl. CPC A61K 35/742; A23K 10/18; A23K 50/10; A61K 35/741; A61K 35/747; C12N 1/205; A23V 2400/117; A23V 2400/617; C12R 2001/01; C12R 2001/10; C12R 2001/125; C12R 2001/225

(57) ABSTRACT

The present disclosure generally relates to compositions and methods for inhibiting pathogenic growth, thereby reducing the severity of diarrhea episodes and also improving health and growth performance of ruminants. More specifically, the disclosure relates to compositions and methods for inhibiting pathogenic growth through the use of a novel combination of probiotic strains.

### FIELD

The present disclosure generally relates to compositions and methods for reducing the severity of diarrhea episodes using a novel combination of probiotic strains, thereby improving health and growth performance of ruminants. Also, the disclosure relates to compositions and methods for inhibiting pathogenic growth using a novel combination of probiotic strains.

### BACKGROUND

Ingestion of pathogens, especially of bacterial origin, but also including viruses and other disease-causing microorganisms, is a common problem in most livestock animals. Pathogens have been known to cause illness in animals, resulting in several deleterious effects, including weight loss, diarrhea, abdominal cramping, and renal failure. For animals that are immunosuppressed or malnourished, even just the effects of diarrhea can be fatal. Therefore, the well-being of the animals is very important, and any illness could potentially affect health and growth performance of the animal, thereby influencing productivity and profitability of the operation. This is especially important in relation to beef cattle where final body weight of the animal at slaughter and carcass weight are of high economic importance.

Pathogens are often transferred between animals and often animal-to-animal transfer cannot be prevented. The most common solution to this problem has been to provide antibiotics to the animals, but besides being costly, this solution can also result in the generation of antibiotic-resistant strains of bacteria, leading to a general agreement in the field that the excessive utilization of antibiotics should be avoided. This phase-out of antibiotics has resulted in an increased need for cost-effective feed additives with high efficiency to support health of the livestock herd.

The utilization of Bacillus-based probiotics in the diets of swine and poultry have yielded beneficial health and production effects. More specifically, Bacillus spp. have gained a greater importance in the feed industry because spores are heat stable and can survive the pelletizing process of animal feed.

WO2018/167171 describes a Bacillus subtilis strain which has sensitivity for ampicillin, vancomycin, gentamycin, kanamycin, streptomycin, erythromycin, clindamycin, tetramycin and chloramphenicol and has inhibitory activity against Escherichia coli and Clostridium perfringens.

WO2004/030624 describes the use of lactic acid producing bacterium either alone or in combination with a lactate-utilizing bacterium to treat or prevent an intestinal pathogenic infection in ruminants, the pathogen preferably being E. coli O157:H7, thereby minimizing the risk of transfer to humans through the meat of the treated animals.

WO2011/115306 describes the use of Bacillus subtilis either alone or in combination with Lactobacillus and Bifidobacterium to improve feed utilization efficiency and milk yield of a ruminant. This is achieved by improving ruminal fermentation.

The prior art cited above focuses either on monogastric animals and their health, or on transfer of pathogens from the meat of ruminant production animals to humans, or on improving ruminal fermentation.

However, there is still a need for probiotic strains and specific mixtures of probiotic strains which can be used to improve health and growth performance of production animals, especially within the beef cattle segment.

### SUMMARY

The present disclosure provides a composition comprising a novel combination of probiotic strains which have a proven effect on the health and growth performance of ruminants. Especially for beef cattle, growth performance is particularly important.

Compositions comprising the novel combination of probiotic strains of the disclosure may be fed to a ruminant, e.g., as Direct Fed Microbial (DFM), as an animal feed additive, in a premix, or incorporated in the animal feed directly.

The novel combination of probiotic strains of the disclosure may be added to the feed during production, after production by the supplier or by the producer mixing the strains with the feed to be fed to the animal, just prior to providing the feed to the animal.

The clostridial diseases are caused by bacteria which belong to a group of organisms called Clostridium. These bacteria are important in farm animal production because they can cause significant disease problems. The Clostridium group of organisms produces very potent exotoxins (poisons), which are damaging to animals.

Bovine enterotoxemia is a major cause of mortality in veal calves. Predominantly veal calves of beef cattle breeds are affected, and losses due to enterotoxemia may account for up to 20% of total mortality. Clostridium perfringens type A is considered to be the major causative agent.

Typical treatments for calves with milder clinical signs consist largely of antibiotics (especially penicillin) and the use of C. perfringens antitoxin products. Several injectable antitoxin preparations that contain specific antibodies directed against toxins produced by C. perfringens are currently available, however there is no effective treatment present at the moment. At present, prevention of clostridial diseases relies largely on vaccination, however such vaccination is reported to only be partially effective in prevention. The whole herd should be vaccinated following the recommendations on the vaccine label. This is a costly procedure to undertake and full prevention cannot be achieved.

The purpose of the in vitro studies described in the Examples was to investigate the inhibition effect of a novel combination of probiotics according to the disclosure on C. perfringens type A and type C. The purpose of the in vivo studies described in the Examples was to investigate the subsequent effect of feed additives comprising a novel combination of probiotics according to the disclosure on the pathogenesis of C. perfringens type A in a challenge trial in beef calves. Other in vivo studies in beef cattle described in the Examples focused on growth performance of feedlot beef cattle after having received feed additives comprising a novel combination of probiotics according to the disclosure.

This disclosure describes the benefits of a novel combination of probiotic strains (Lactobacillus animalis, Propionibacterium freudenreichii, Bacillus licheniformis, and Bacillus subtilis) in inhibiting C. perfringens type A and type C growth and improving the health and growth performance of beef cattle. These benefits have been assessed in (i) an in vitro assay demonstrating the inhibitory effect of the novel combination of probiotic strains on growth of C. perfringens type A and type C (Example 1), and (ii) a Clostridium perfringens type A challenge in vivo trial, where beef calves receiving the novel combination of probiotics of the disclosure had reduced number of days of diarrhea (Example 2), and (iii) an in vivo feedlot performance trial where finishing beef animals receiving a high-concentrate diet and fed the novel combination of probiotics of the disclosure were heavier at slaughter, had a greater average daily gain (ADG) and feed efficiency (FE) during the feedlot period, as well as greater carcass weight following slaughter (Example 3).

These results demonstrate that the aforementioned novel combination of probiotic strains improves health and performance of the beef cattle herd, thereby providing a cost-effective and easy to administer alternative to the traditional approach of treatment and minimization of C. perfringens infection using antibiotics and vaccination, while at the same time improving growth performance in beef cattle.

### Definitions

In general, the terms and phrases used herein have their art-recognized meaning, which can be found by reference to standard textbooks, journal references and context known to those skilled in the art. The following definitions are provided to clarify their specific use in context of the present disclosure.

As used herein, the singular forms “a”, “an” and “the” are intended to include the plural forms as well, unless the context clearly indicates otherwise.

As used herein, the term “and/or” is intended to mean the combined (“and”) and the exclusive (“or”) use, i.e. “A and/or B” is intended to mean “A alone, or B alone, or A and B together”.

Animal feed: As used herein the term “animal feed” refers to any compound, preparation, or mixture suitable for, or intended for intake by an animal. Animal feed for a ruminant comprises concentrates as well as for example vitamins, minerals, enzymes, amino acids and/or other feed ingredients (such as in a premix). The animal feed may further comprise forage. Examples of ruminant feed are given in Example 3.

Composition: As used herein the term “composition” refers to a composition comprising a carrier and at least one bacterial strain as described herein. The compositions described herein may be used in a Direct Fed Microbial (DFM), an animal feed additive, premix or Total Mixed Ration (TMR).

Concentrate: As used herein the term “concentrate” means a feed with high protein and energy concentrations, such as fish meal, molasses, oligosaccharides, seeds and grains (either whole or prepared by crushing, milling, etc. from, e.g., corn, oats, rye, barley, sorghum, wheat), oilseed press cake (e.g. from cottonseed, safflower, sunflower, soybean (such as soybean meal), rapeseed/canola, peanut or groundnut), palm kernel cake, yeast-derived material and distillers grains (such as wet distillers grains (WDS) and dried distillers grains with solubles (DDGS)).

Inhibit the growth of C. perfringens: As used herein the term “inhibit the growth of C. perfringens” means a method and/or composition that partly or completely inhibits C. perfringens infections in an animal. Accordingly, the term “inhibit the growth of C. perfringens” means the C. perfringens infections are reduced or completely eliminated and the overall health of the animal is improved.

Direct Fed Microbial: As used herein the term “direct fed microbial” or “DFM” means live microorganisms including spores which, when administered in adequate amounts, confer a benefit, such as improved digestion or health, on the animal to which it is administered.

Effective amount/concentration/dosage: As used herein the terms “effective amount”, “effective concentration” or “effective dosage” are defined as the amount, concentration or dosage of the bacterial strain(s) sufficient to improve health and performance (feed digestion or efficiency) of an animal, as illustrated in the examples.

The actual effective dosage in absolute numbers depends on factors including: the state of health of the animal in question, and other ingredients present. The “effective amount”, “effective concentration” or “effective dosage” of the bacterial strains may be determined by routine assays known to those skilled in the art in view of the guidance provided herein. An example of an effective amount for ruminants is given in Examples 2 and 3.

Feeding an animal: As used herein the terms “feeding an animal” or “fed to an animal” means that the composition of the present disclosure is administered orally to the animal in an effective amount. The oral administration may be repeated, e.g., one or more times daily over a specified period of time, such as several days, one week, several weeks, one month or several months. Feeding of ruminants can, e.g., be performed as described in Examples 2 and 3. Accordingly, the terms “feeding” or “fed” mean any type of oral administration, such as administration via an animal feed or via drinking water or, in certain circumstances, by oral gavage or aerosol spray.

Isolated: As used herein the term “isolated” means that the bacterial strains described herein are in a form or environment which does not occur in nature, i.e., the strain is at least partially removed from one or more or all of the naturally occurring constituents with which it is associated in nature.

Minimize C. perfringens infections: As used herein the term “minimize C. perfringens infections” means a method and/or composition that minimizes development of a C. perfringens infection in an animal.

Body Weight Gain: As used herein the Body Weight Gain of an animal is the increase of weight of the animal over a specified period of time. An example of average Weight Gain determination is given in Example 3. Weight gain is also described using the term “Average Daily Gain (ADG)”. An example of average daily gain is given in Example 3. As used herein the term “Average Daily Gain” and “ADG” means the same and are used interchangeably. The term “Average Daily Gain” means the amount of weight gained by one animal within one day, i.e. within 24 hours.

General impression: As used herein the term “General impression” is a measure of the overall general impression of a given animal at a given day, and is assessed by methodologies known in the field.

Dry Matter intake (DMI): As used herein the term “Dry Matter Intake” and the term “DMI” means the same and are used interchangeably. The term “Dry Matter Intake” means the amount of feed consumed, excluding water content.

Feed efficiency (FE): As used herein the term “Feed efficiency” and the term “FE” means the same and are used interchangeably. By “increased feed efficiency” it is meant that the use of the feed additive composition of the disclosure in feed results in a lower amount of feed being required for the animal to gain 1 kg of body weight compared to the amount of feed required to increase the same 1 kg of body weight of the animal when the feed does not comprise said feed additive composition of the disclosure.

Hot carcass weight: As used herein the term “Hot carcass weight” means the value obtained by weighing the edible parts of the carcass after slaughter, removing bones, internal organs, viscera, etc. It is thus a weight lower than the total body weight of the slaughtered animal and a measure of the amount of edible meat on the animal.

### BRIEF DESCRIPTION OF THE FIGURES

FIG. 1 shows the effect of the probiotic mixture of the disclosure against C. perfringens type A (5 replicates). Clear zones show inhibition of the pathogen by the tested product (spotted in the wells).

FIG. 2 shows the effect of the probiotic mixture of the disclosure against C. perfringens type C (5 replicates). Clear zones show inhibition of the pathogen by the tested product (spotted in the wells).

FIG. 3 shows the diameter of the inhibition zones of the probiotic mixture of the disclosure (Treat-A) and competitor #1 (B. subtilis) against C. perfringens type A at 24 h post-probiotic incubation.

FIG. 4 shows the diameter of the inhibition zones of the probiotic mixture of the disclosure (Treat-A) and competitor #2 (L. salivarius) against C. perfringens types A (FIG. 4A) and C (FIG. 4B) at 24 h post-probiotic incubation.

FIG. 5 shows the diameter of the inhibition zones of the probiotic mixture of the disclosure (Treat-A) and competitors #3 (L. animalis) and #4 (A. acidipropionici) against C. perfringens types A (FIG. 5A) and C (FIG. 5B) at 24 h post-probiotic incubation.

FIG. 6 shows the diameter of the inhibition zones of the probiotic mixture of the disclosure (Treat-A) against Salmonella Heidelberg at 24 h post-probiotic incubation.

FIG. 7 reports the number of days in which animals were considered having an abnormal score, according to the health and diarrhea score, i.e. the number of days where the score is above 0.

FIG. 8 shows transepithelial electrical resistance (TEER) and area under the curve (AUC) across intestinal epithelial cell monolayers exposed or not to the probiotic mixture of the disclosure (Treat-A) and pro-inflammatory cytokines (TNF-α and IFN-γ).

FIG. 9 shows transepithelial electrical resistance (TEER) across intestinal epithelial cell monolayers exposed or not to the probiotic mixture of the disclosure (Treat-A) and Clostridium perfringens type A (ATCC 13124).

### DETAILED DESCRIPTION

Since pathogens are known to populate many distinct areas of animals' digestive tracts, it has been found to be more beneficial to supply and potentiate microorganisms that occur naturally in those areas and which are effective for inhibiting pathogenic growth throughout the digestive tract, such as the rumen, small intestine, and large intestine. The present disclosure identifies such naturally occurring microorganisms suitable for serving this purpose. The present disclosure exploits the natural interaction of certain microorganisms (i.e., probiotics) with some pathogenic microorganisms, with the goal of reducing the load of the latter. The probiotics in the compositions of the disclosure may exhibit multifaceted modes of action. These actions range from complex actions, such as acting as or producing bactericides, to simply competing with the pathogen by using more nutrients, and/or competing by binding sites in the digestive tract, thus preventing pathogens from becoming established within the gastrointestinal tract of treated ruminants. The present disclosure is thus capable of achieving these advantageous actions without administering antibiotics and like substances to the ruminants.

The present disclosure includes a method of inhibiting a gastro-intestinal pathogenic infection in a ruminant comprising administering to said ruminant a composition that inhibits the development of clinical signs of disease associated with said pathogenic bacterium, said composition comprising four different probiotic strains. and may include assessing the effects of pathogen reduction by said composition.

In another embodiment of the present disclosure, a method of increasing average daily weight gain of a ruminant is also described, comprising administering to said ruminant a composition that comprises four different probiotic strains, and optionally assessing the effects on average daily weight gain by said composition.

The probiotic strains of the present disclosure described herein are isolated, i.e., present in a form or environment which does not occur in nature, and have a synergistic effect when used in combination as described herein.

In one embodiment of the disclosure said probiotic strains in said composition are Lactobacillus animalis, Propionibacterium freudenreichii, Bacillus subtilis and Bacillus licheniformis.

In one embodiment of the disclosure said composition comprising probiotic strains is administered to said ruminant in an amount to provide a total of between 1×10⁸ to 1×10¹¹ CFU/head/day, such as between 1×10⁸ to 1×10¹⁰ CFU/head/day, such as between 1×10⁹ to 1×10¹⁰ CFU/head/day, such as between 5×10⁹ to 5×10¹⁰ CFU/head/day.

In a preferred embodiment of the disclosure, said composition comprising probiotic strains is administered to said ruminant in an amount to provide a total of between 6×10⁹ to 8×10⁹ CFU/head/day, such as 6×10⁹ CFU/head/day, such as 7×10⁹ CFU/head/day, such as 8×10⁹ CFU/head/day.

In the most preferred embodiment of the disclosure, said composition comprising probiotic strains is administered to said ruminant in an amount to provide a total of 6×10⁹ CFU/head/day.

The term “CFU/head/day” relates to the amount of probiotic strains administered to each ruminant per day. This term thus excludes carriers such as calcium carbonate, anti-caking agents such as aluminum silicates and kieselguhr (diatomaceous earth) and other components which optionally may be present in the composition.

Compositions of the present disclosure include at least the four species of probiotic strains of the disclosure and at least one carrier and/or other components that make the composition suitable for feeding an animal or as an additive for drinking water.

In one embodiment of the disclosure, said composition comprising probiotic strains, comprises about 50% Bacillus strains and about 50% non-Bacillus strains. Typically, the majority of the Bacillus strains present in the composition of the disclosure is Bacillus licheniformis, such that in one embodiment of the present disclosure Bacillus licheniformis accounts for 75% of the total Bacillus strains in the composition of the disclosure.

The non-Bacillus strains present in the composition of the disclosure are selected from the group consisting of Lactobacillus and Propionibacterium, such as Lactobacillus animalis and Propionibacterium freudenreichii.

In one preferred embodiment of the present disclosure the ratio in colony forming unit (CFU) between said probiotic strains present in the composition of the disclosure is 1:1:1:3 Lactobacillus animalis:Propionibacterium freudenreichii:Bacillus subtilis:Bacillus licheniformis.

In one embodiment of the present disclosure the composition of the present disclosure comprises probiotic strains of four different species: Lactobacillus animalis, Propionibacterium freudenreichii, Bacillus subtilis and Bacillus licheniformis, wherein a ratio in colony forming unit (CFU) of said probiotic strains of said species is 1:1:1:3, respectively.

In one preferred embodiment of the present disclosure the microorganism components of the composition of the present disclosure consists of four different probiotic strains, one each of Lactobacillus animalis, Propionibacterium freudenreichii, Bacillus subtilis and Bacillus licheniformis, present in a ratio in colony forming unit (CFU) of said probiotic strains of 1:1:1:3, respectively. In such embodiments, the composition does not include any other microorganism components, but may include other components, such as one or more carriers and/or one or more feed ingredients.

In one embodiment of the present disclosure the Lactobacillus animalis strain present in said composition is the LA51 strain deposited as DSM33570.

In one embodiment of the present disclosure the Propionibacterium freudenreichii strain present in said composition is the PF24 strain deposited as DSM34127.

In one embodiment of the present disclosure the Bacillus subtilis strain present in said composition is the strain deposited as DSM32324.

In one embodiment of the present disclosure the Bacillus licheniformis strain present in said composition is the strain deposited as DSM17236.

In one embodiment of the present disclosure the composition comprises four different probiotic strains consisting of the Lactobacillus animalis strain deposited as DSM33570, the Propionibacterium freudenreichii deposited as DSM34127, the Bacillus subtilis deposited as DSM32324, and the Bacillus licheniformis deposited as DSM17236, optionally in a ratio in colony forming unit (CFU) of said probiotic strains of 1:1:1:3, respectively.

In one embodiment of the present disclosure the microorganism components of the composition consists of four different probiotic strains consisting of the Lactobacillus animalis strain deposited as DSM33570, the Propionibacterium freudenreichii deposited as DSM34127, the Bacillus subtilis deposited as DSM32324, and the Bacillus licheniformis deposited as DSM17236, optionally in a ratio in colony forming unit (CFU) of said probiotic strains of 1:1:1:3, respectively. In such embodiments, the composition does not include any other microorganism components, but may include other components, such as one or more carriers and/or one or more feed ingredients.

The probiotic strains of the composition of the present disclosure may be provided in the form of spores and/or bacterial cells. In one embodiment of the present disclosure the Bacillus strains (Bacillus subtilis and Bacillus licheniformis) are provided in the form of spores, while the Lactobacillus animalis and the Propionibacterium freudenreichii are provided in the form of bacterial cells.

In one embodiment of the present disclosure the composition of the present disclosure comprises an amount of Lactobacillus animalis strain DSM33570 to provide between 0.15×10⁸ and 0.15×10¹¹ CFU/day of Lactobacillus animalis strain DSM33570, preferably between 1×10⁹ and 1×10¹⁰ CFU/day Lactobacillus animalis strain DSM33570. In a preferred embodiment the composition of the present disclosure comprises an amount of Lactobacillus animalis strain DSM33570 to provide 1×10⁹ CFU/day of Lactobacillus animalis strain DSM33570.

In one embodiment of the present disclosure the composition of the present disclosure comprises an amount of Propionibacterium freudenreichii strain DSM34127 to provide between 0.15×10⁸ and 0.15×10¹¹ CFU/day of Propionibacterium freudenreichii strain DSM34127, preferably between 1×10⁹ and 1×10¹⁰ CFU/day Propionibacterium freudenreichii strain DSM34127. In a preferred embodiment the composition of the present disclosure comprises an amount of Propionibacterium freudenreichii strain DSM34127 to provide 1×10⁹ CFU/day of Propionibacterium freudenreichii strain DSM34127.

In one embodiment of the present disclosure the composition of the present disclosure comprises an amount of Bacillus subtilis strain DSM32324 to provide between 0.15×10⁸ and 0.15×10¹¹ CFU/day of Bacillus subtilis strain DSM32324, preferably between 1×10⁹ and 1×10¹⁰ CFU/day Bacillus subtilis strain DSM32324. In a preferred embodiment the composition of the present disclosure comprises an amount of Bacillus subtilis strain DSM32324 to provide 1×10⁹ CFU/day of Bacillus subtilis strain DSM32324.

In one embodiment of the present disclosure the composition of the present disclosure comprises an amount of Bacillus licheniformis strain DSM17236 to provide between 0.45×10⁸ and 0.45×10¹¹ CFU/day of Bacillus licheniformis strain DSM17236, preferably between 3×10⁹ and 3×10¹⁰ CFU/day Bacillus licheniformis strain DSM17236. In a preferred embodiment the composition of the present disclosure comprises an amount of Bacillus licheniformis strain DSM17236 to provide 3×10⁹ CFU/day of Bacillus licheniformis strain DSM17236.

In one embodiment of the present disclosure the composition comprises amounts of the probiotic strains to provide 1×10⁹ CFU/day of the Lactobacillus animalis strain deposited as DSM33570, 1×10⁹ CFU/day of the Propionibacterium freudenreichii deposited as DSM34127, 1×10⁹ CFU/day of the Bacillus subtilis deposited as DSM32324 and 3×10⁹ CFU/day of the Bacillus licheniformis deposited as DSM17236.

In one embodiment of the present disclosure the microorganism components of the composition consists of amounts of the probiotic strains to provide 1×10⁹ CFU/day of the Lactobacillus animalis strain deposited as DSM33570, 1×10⁹ CFU/day of the Propionibacterium freudenreichii deposited as DSM34127, 1×10⁹ CFU/day of the Bacillus subtilis deposited as DSM32324 and 3×10⁹ CFU/day of the Bacillus licheniformis deposited as DSM17236. In such embodiments, the composition does not include any other microorganism components, but may include other components, such as one or more carriers and/or one or more feed ingredients.

The relevant probiotic strains are provided in a commercially relevant form known to the skilled person. Accordingly, in an embodiment, the probiotic strains of the composition are present in a dried (e.g., spray dried or freeze dried) or frozen form. The different probiotic strains present in the composition of the present disclosure may be present in different forms in the mixture, e.g. some may be spray dried and some may be freeze dried. The composition may be provided in any suitable form such as in the form of a liquid e.g., a gel, a slurry, etc., or a in the form of a solid, e.g., a powder or a pellet.

For compositions in the form of a premix, the probiotic strains of the disclosure may be added to a carrier to make a mineral-vitamin mixture (premix), which may then be added to an animal feed at a desired inclusion rate.

Alternatively, for compositions in the form of an animal feed, the probiotic strains of the of the disclosure may be formulated with animal feed ingredients, as illustrated below. Such combinations of the composition of the disclosure and animal feed ingredients optionally may be in the form of pellets that are extruded through standard pelleting processes.

The disclosure also provides a method for producing an animal feed, animal feed additive, or premix comprising adding the four species of probiotic strains of the disclosure to an animal feed or relevant components thereof.

In one embodiment, the present disclosure provides an animal feed, animal feed additive, or premix comprising the probiotic strains of the disclosure, and further comprising one or more concentrate(s), vitamin(s), mineral(s), enzyme(s), amino acid(s) and/or other feed ingredient(s).

In one embodiment the animal feed, animal feed additive, or premix comprises the composition of the disclosure comprising the Lactobacillus animalis strain deposited as DSM33570, the Propionibacterium freudenreichii deposited as DSM34127, the Bacillus subtilis deposited as DSM32324 and the Bacillus licheniformis deposited as DSM17236, and further comprising one or more concentrate(s), vitamin(s), mineral(s), enzyme(s), amino acid(s) and/or other feed ingredient(s).

In a preferred embodiment the animal feed, animal feed additive or premix comprises the composition of the disclosure consisting of the Lactobacillus animalis strain deposited as DSM33570, the Propionibacterium freudenreichii deposited as DSM34127, the Bacillus subtilis deposited as DSM32324 and the Bacillus licheniformis deposited as DSM17236, and further comprising one or more concentrate(s), vitamin(s), mineral(s), enzyme(s), amino acid(s) and/or other feed ingredient(s).

In a specific embodiment, the animal feed is a Total Mixed Ration (TMR) comprising 0 to 80% forage source, such as hay, silage or pasture. Generally, forage comprises edible parts of a plant (other than separated grain) that can provide feed for animals or can be harvested for feeding animals. As an example, the TMR comprises 0-80% maize; and/or 0-80% sorghum; and/or 0-70% wheat; and/or 0-70% barley; and/or 0-30% oats; and/or 0-40% soybean meal.

In one embodiment, the composition of the present disclosure comprising the four species of probiotic strains are mixed with forage, concentrate, and optionally other feed components to obtain a TMR.

In a further embodiment, the composition of the present disclosure comprising the four species of probiotic strains is mixed with concentrate, vitamins and/or minerals to obtain a premix. This premix can be mixed with a final diet to obtain a TMR. In a further embodiment, the TMR and the composition of the present disclosure comprising the four species of probiotic strains is mixed with one or more enzymes. In a further embodiment, the composition of the present disclosure comprising the four species of probiotic strains are mixed with other feed ingredients, such as one or more of coloring agents, stabilizers, growth improving additives and aroma compounds/flavorings, saturated or polyunsaturated fatty acids (PUFAs), essential oils, anti-oxidants, anti-microbial peptides, anti-fungal polypeptides and amino acids.

In a particular embodiment, the animal feed components consists of or comprises milk (e.g., from cow, goat, sheep), e.g., for feeding of calves. In another particular embodiment, the animal feed components consists of or comprises milk replacement, e.g., for feeding of calves. In one embodiment the composition of the present disclosure comprising the four species of probiotic strains is mixed with water, milk or milk replacer for feeding of calves.

In another embodiment, the animal feed components may include one or more vitamins, such as one or more fat-soluble vitamins and/or one or more water-soluble vitamins. In another embodiment, the animal feed components may optionally include one or more minerals, such as one or more trace minerals and/or one or more macro minerals. Usually fat- and water-soluble vitamins, as well as trace minerals, form part of a so-called premix intended for addition to the feed, whereas macro minerals are usually separately added to the feed. Non-limiting examples of fat-soluble vitamins include vitamin A, vitamin D3, vitamin E, and vitamin K, e.g., vitamin K3. Non-limiting examples of water-soluble vitamins include vitamin B12, biotin and choline, vitamin B1, vitamin B2, vitamin B6, niacin, folic acid and pantothenate, e.g., Ca-D-pantothenate. Non-limiting examples of trace minerals include boron, cobalt, chloride, chromium, copper, fluoride, iodine, iron, manganese, molybdenum, selenium and zinc. Non-limiting examples of macro minerals include calcium, magnesium, potassium and sodium.

The animal feed, animal feed additive or premix of the disclosure may also comprise at least one enzyme selected from the group comprising of phytase (EC 3.1.3.8 or 3.1.3.26); xylanase (EC 3.2.1.8); galactanase (EC 3.2.1.89); alpha-galactosidase (EC 3.2.1.22); protease (EC 3.4); phospholipase A1 (EC 3.1.1.32); phospholipase A2 (EC 3.1.1.4); lysophospholipase (EC 3.1.1.5); phospholipase C (3.1.4.3); phospholipase D (EC 3.1.4.4); amylase such as, for example, alpha-amylase (EC 3.2.1.1); lysozyme (EC 3.2.1.17); cellulase (EC 3.2.1.4); and beta-glucanase (EC 3.2.1.6), or any mixture thereof.

The animal feed, animal feed additive or premix of the disclosure may further comprise one or more added amino acids. Examples of amino acids which are used in animal feed are rumen-protected or not rumen-protected lysine, alanine, beta-alanine, threonine, methionine and tryptophan. The animal feed, animal feed additive or premix of the disclosure may further comprise coloring stabilizers, agents, growth improving additives and aroma compounds/flavorings, polyunsaturated fatty acids (PUFAs), essential oils, anti-oxidants, anti-microbial peptides and anti-fungal polypeptides. Examples of coloring agents are carotenoids such as beta-carotene, astaxanthin, and lutein. Examples of aroma compounds/flavorings are creosol, anethol, deca-, undeca- and/or dodeca-lactones, ionones, irone, gingerol, piperidine, propylidene phthalide, butylidene phthalide, capsaicin and tannin. Examples of saturated fatty acids are C16 and C18, such as palmitic and oleic acids, and polyunsaturated fatty acids are C18, C20 and C22 polyunsaturated fatty acids, such as linoleic, linolenic, arachidonic acid, docosahexaenoic acid, eicosapentaenoic acid and gamma-linoleic acid. Examples of reactive oxygen generating species are chemicals such as perborate, persulphate, or percarbonate; and enzymes such as an oxidase, an oxygenase or a synthetase.

In one embodiment the animal feed, animal feed additive or premix comprises one or more coccidiostats.

In one embodiment the animal feed, animal feed additive or premix further comprises a carrier. The carrier can comprise one or more of the following compounds: water, glycerol, ethylene glycol, 1,2-propylene glycol or 1,3-propylene glycol, sodium chloride, sodium benzoate, potassium sorbate, sodium sulfate, potassium sulfate, magnesium sulfate, sodium thiosulfate, calcium carbonate, sodium citrate, dextrin, maltodextrin, glucose, sucrose, sorbitol, lactose, whey, whey permeate, wheat flour, wheat bran, corn gluten meal, starch and cellulose.

In an embodiment, the animal feed, animal feed additive or premix further comprises one or more additional microorganisms. In a particular embodiment, the animal feed, animal feed additive or premix further comprises a bacterium from one or more of the following genera: Lactobacillus, Lactococcus, Streptococcus, Bacillus, Pediococcus, Enterococcus, Leuconostoc, Carnobacterium, Propionibacterium, Bifidobacterium, Clostridium and Megasphaera or any combination thereof.

In a particular embodiment, the animal feed, animal feed additive or premix further comprises a bacterium from one or more of the following strains of Bacillus amyloliquefaciens, Bacillus subtilis, Bacillus pumilus, Bacillus polymyxa, Bacillus licheniformis, Bacillus megaterium, Bacillus coagulans, Bacillus circulans, Bacillus simplex, Bacillus mojavensis, Bacillus safensis, Bacillus simplex, Bacillus atrophaeus, Bacillus methylotrophicus, Bacillus siamensis, Bacillus vallismortis, Bacillus tequilensis or any combination thereof.

In a particular embodiment, the animal feed, animal feed additive or premix further comprises one or more types of yeast. The one or more types of yeast can be selected from the group consisting of Saccharomycesceae, Saccharomyces (such as S. cerevisiae and/or S. boulardii), Kluyveromyces (such as K. marxianus and K. lactis), Candida (such as C. utilis, also called Torula yeast), Pichia (such as P. pastoris), Torulaspora (such as T. delbrueckii), Phaffia yeasts and Basidiomycota.

The composition of the present disclosure may additionally comprise cryoprotectants, lyoprotectants, antioxidants, nutrients, fillers, flavorants or mixtures thereof. The composition may be in frozen or freeze-dried form. The composition preferably comprises one or more of cryoprotectants, lyoprotectants, and/or nutrients, antioxidants more preferably cryoprotectants, lyoprotectants and/or antioxidants and most preferably cryoprotectants or lyoprotectants, or both. Use of protectants such as cryoprotectants and lyoprotectants known to a skilled person in the art. Suitable cryoprotectants or lyoprotectants include mono-, di-, tri- and polysaccharides (such as glucose, mannose, xylose, lactose, sucrose, trehalose, raffinose, maltodextrin, starch and gum arabic (acacia) and the like), polyols (such as erythritol, glycerol, inositol, mannitol, sorbitol, threitol, xylitol and the like), amino acids (such as proline, glutamic acid), complex substances (such as skim milk, peptones, gelatin, yeast extract) and inorganic compounds (such as sodium tripolyphosphate). Suitable antioxidants include ascorbic acid, citric acid and salts thereof, gallates, cysteine, sorbitol, mannitol, maltose. Suitable nutrients include sugars, amino acids, fatty acids, minerals, trace elements, vitamins (such as vitamin B-family, vitamin C). The composition may optionally comprise further substances including fillers (such as lactose, maltodextrin) and/or flavorants.

Animal diets can, e.g., be manufactured as mash feed (non-pelleted) or pelleted feed. Typically, the milled feedstuffs are mixed and sufficient amounts of essential vitamins and minerals are added according to the specifications for the species in question. The bacteria cultures and optionally enzymes can be added as solid or liquid formulations. For example, for mash feed a solid or liquid culture formulation may be added before or during the ingredient mixing step. For pelleted feed the (liquid or solid) composition of the disclosure comprising the four species of probiotic strains may be added to the pelleted food after the pelleting step. Typically, a liquid composition of the disclosure comprises the four species of probiotic strains optionally with a polyol, such as glycerol, ethylene glycol or propylene glycol, and is added after the pelleting step, such as by spraying the liquid formulation onto the pellets. The probiotic strains may also be incorporated in an animal feed additive or premix.

The composition according to the disclosure may be used for the minimization or control of a bacterial colonization or infection by pathogens, including gas producing pathogens, e.g., Clostridium spp., such as Clostridium difficile, Clostridium novyi, Clostridium perfringens, or Clostridium septicum. In a preferred embodiment, the composition according to the disclosure may be used for the minimization or control of Clostridium perfringens type A and type C.

Another aspect of the disclosure relates to a method for feeding an animal comprising administering the composition of the disclosure comprising the four species of probiotic strains to an animal, in particular a ruminant. Ruminants include cow, cattle, sheep, deer, and goat.

Ruminants have a fundamentally different digestive system than monogastric animals, and it therefore cannot be concluded that a composition designed for a monogastric animal and with proven effect in same would also be suitable for and have an effect in a ruminant.

The digestive system of animals is involved in the mechanical and chemical digestion of food, absorption of nutrients, and elimination of indigestible materials from the body. The main difference between monogastric and ruminant digestive system is that digestion in the monogastric digestive system mainly occurs in the stomach, whereas digestion in the ruminant digestive system is a foregut fermenter type digestion. The monogastric digestive system is composed of a single stomach while the ruminant digestive system is composed of four stomachs (reticulum, rumen, omasum, and abomasum). Monogastric digestive systems mainly occur in omnivores and carnivores, while ruminants are herbivores.

The monogastric digestive system refers to the organ system which helps the digestion of both animal and plant materials. It is called monogastric since this digestive system is composed of a single stomach. Human, horse, swine, fowl, dog, bird and rabbit-like animals have a monogastric digestive system. The digestion begins with the entering of feed to the mouth. Both chemical and mechanical digestion starts at the mouth. Saliva contains enzymes to digest carbohydrates. The esophagus is the passage that leads feed to the stomach. Various enzymes are secreted into the lumen of the stomach to digest proteins in the feed. Animals with a monogastric digestive system mainly take animal tissues as food. Their diet is easy to digest. Thus, a single stomach is enough for the purpose. Small intestine mainly absorbs the nutrients from the digested feed. The large intestine absorbs water from the indigestible materials.

The ruminant digestive system refers to the organ system in which the digestion of plant materials occurs. Cow, cattle, sheep, deer, and goat are examples of the animals having a ruminant digestive system. The top jaw of ruminant animals lacks teeth in the front, but instead, a hard pad of skin is present, which is called the dental pad. Other than the basic anatomy of an animal digestive system, the ruminant digestive system is composed of four stomachs. They are rumen, reticulum, omasum, and abomasum. The first three stomachs, the rumen, reticulum, and omasum, are involved in the breaking down of plant fibers and digestion of non-fibrous compounds. The population of microflora is involved in this process. It breaks down cellulose and starch, for example, through fermentation, producing volatile fatty acids such as acetate, butyrate and propionate. These volatile fatty acids are utilized by the ruminant as an energy source. Digestive enzymes are secreted in the fourth stomach called the abomasum. Therefore, fermentation occurs before the digestion of the feed in ruminant animals. Hence, this process is called the foregut fermentation. Furthermore, ruminant animals chew the partly digested food or cud by returning them from the first stomach. Small intestine and large intestine of ruminants are similar to the monogastric digestive system. However, ruminants comprise a large caecum for further digestion of the fibers.

In one embodiment of the present disclosure, the composition of the present disclosure is used in a method of inhibiting an gastrointestinal pathogenic infection and/or a method of increasing average daily weight gain in a ruminant.

In a preferred embodiment the ruminant is cattle. Cattle can further be divided into beef cattle and dairy cattle.

Dairy cattle breeds have been bred over hundreds of years to produce large amounts of milk. This is their key difference from beef breeds, as they produce milk in excess of what their calf needs. Dairy cows have large, pronounced udders and can produce up to 12 gallons of milk per day (approximately). Beef cattle are stockier than dairy cattle and the cows of beef breeds only produce enough milk for their calves (about one to two gallons per day), unlike dairy cattle.

Beef cattle are like weightlifters. They are stocky because their energy goes toward building muscle and storing fat. Dairy cows are more like marathon runners. They are thin and lean with a more angular shape. Their energy goes into producing milk rather than building muscle and storing fat. In fact, those two characteristics—producing milk versus producing mass—are usually mutually exclusive in cattle, which is why there's a difference between beef and dairy cattle.

In a preferred embodiment of the present disclosure the ruminant is cattle, preferably beef cattle.

As evidenced in the examples, administration of the composition of the disclosure comprising the four species of probiotic strains described herein improves the gastrointestinal and overall health of the ruminant and provides improved animal performance parameters for the treated ruminants as compared to controls. Animal performance parameters include, but are not limited to, dry matter intake (DMI), average daily weight gain (ADG) and feed efficiency (FE).

In a preferred embodiment of the disclosure, “animal performance” is determined by the body weight gain of the animal and/or by the feed efficiency. By “improved animal performance” it is meant that there is increased body weight gain and/or increased feed efficiency and/or improved average daily gain and/or increased hot carcass weight resulting from the use of animal feed, animal feed additive or premix of the present disclosure in animal feed in comparison to animal feed which does not comprise said animal feed, animal feed additive or premix. Preferably, by “improved animal performance” it is meant that there is increased body weight gain and/or increased hot carcass weight.

An “increased weight gain” refers to an animal having increased body weight on being fed feed comprising a feed composition of the disclosure compared with an animal being fed a feed without said feed composition of the disclosure. Specifically, the Body Weight Gain (BWG) of an animal is the increase of weight of the animal over a specified period of time (in days). In one embodiment, the improvement in BWG is of at least 0.5%, such as at least 1%, such as at least 2%, such as at least 2.5%, such as at least 3%, such as at least 4%, such as at least 5%, such as at least 6%, such as at least 7%, such as at least 8%, such as at least 9%, such as at least 10%.

In one embodiment, the improvement in BWG results in a body weight gain of at least 0.5%, such as at least 0.8%, such as at least 1.2%, such as at least 1.5%, such as at least 1.8%, such as at least 2.0%, such as at least 2.5%, such as at least 3.0%, such as at least 4.0%, such as at least 5.0%, such as at least 6.0%, such as at least 7.0%. In a preferred embodiment, the improvement in BWG results in a weight gain selected from the group consisting of from 1.0% to 5.0%, from 1.5% to 5.0%, from 2.0% to 5.0%, from 2.5% to 5.0%, from 3.0% to 5.0%, from 3.5% to 5.0%, from 4.0% to 5.0%, or from 4.5% to 5.0%.

In another preferred embodiment, the improvement in BWG results in a weight gain selected from the group consisting of from 1.8% to 2.0%, from 2.0% to 2.2%, from 2.2% to 2.4%, from 2.4% to 2.6%, from 2.6% to 2.8%, from 2.8% to 3.0%, from 3.0% to 3.2%, from 3.2% to 3.4%, from 3.4% to 3.6%, from 3.6% to 3.8%, from 3.8% to 4.0%, from 4% to 5%, from 5% to 7%, from 7% to 10%, or any combination thereof.

By “increased feed efficiency” it is meant that the use of the feed additive composition of the disclosure in feed results in a lower amount of feed being required for the animal to gain 1 kg of body weight compared to the amount of feed required to increase the same 1 kg of body weight of the animal when the feed does not comprise said feed additive composition of the disclosure.

In one embodiment, the improvement of feed efficiency (FE) results in an improvement of FE of 3.0% or more than 3.0%, such as more than 3.5%, such as more than 4.0%, such as more than 4.5%, such as more than 5.0%, such as more than 5.5%. In a preferred embodiment, the improvement of FE results in an increased FE of from 3.0% to 7.0%, such as an increased FE of from 3.0% to 6.5%, such as an increased FE of from 4.0% to 6.5%. In a specific embodiment, the improvement of FE results in an increased FE within an interval selected from the group consisting of from 3.0% to 7.0%, from 3.5% to 7.0%, from 4.0% to 7.0%, from 4.5% to 7.0%, from 5.0% to 7.0%, from 5.5% to 7.0%, from 6.0 to 7.0%, or any combination of these intervals.

In another embodiment, the disclosure relates to a method of improving one or more animal performance parameters selected from the group consisting of:
i) increased body weight gain (BWG),
ii) increased feed efficiency (FE),
iii) increased hot carcass weight (HCW),
iv) reduced number of days with diarrhea,
v) increased number of days having normal appearance,
   the method comprising feeding the composition of the present disclosure comprising four the typies of probiotic strains to an animal.

In an embodiment of the present disclosure the method of inhibiting a gastrointestinal pathogenic infection in a ruminant, further comprises assessing the effects of pathogen reduction by said administration of said composition.

In an embodiment of the present disclosure the pathogenic bacterium is Clostridium perfringens. The Clostridium perfringens is in one embodiment type A (DSM756) or C. perfringens type C (NCTC3180).

In one embodiment of the present disclosure the pathogenic bacterium is Salmonella. The Salmonella is in one embodiment Salmonella Heidelberg.

In one embodiment of the present disclosure the method of increasing average daily weight gain of a ruminant, further comprises assessing the effects on average daily weight gain by said administration of said composition. In one embodiment of the present disclosure, the increase in average daily weight gain results in an increased final body weight and hot carcass weight.

### Taxonomy

Lactobacillus animalis is now known as Ligilactobacillus animalis, as described in Zheng et al., Int. J. Syst. Evol. Microbiol. DOI 10.1099/ijsem.0.004107. The two different names are used interchangeably herein.

### DEPOSIT AND EXPERT SOLUTION

The applicant requests that a sample of the deposited microorganisms stated below may only be made available to an expert, subject to available provisions governed by Industrial Property Offices of States Party to the Budapest Treaty, until the date on which the patent is granted.

Table 1: Deposits made at a Depositary institution having acquired the status of international depositary authority under the Budapest Treaty on the International Recognition of the Deposit of Microorganisms for the Purposes of Patent Procedure: Leibniz Institute DSMZ-German Collection of Microorganisms and Cell Cultures Inhoffenstr. 7B, 38124 Braunschweig, Germany being Lactobacillus animalis, Propionibacterium freudenreichii, Bacillus subtilis and Bacillus licheniformis

| Strain | Accession No. | Deposit date (D. M. Y) |
|---|---|---|
| Lactobacillus animalis/ | DSM 33570 | 8 Jul. 2020 |
| Ligilactobacillus animalis | | |
| Propionibacterium | DSM 34127 | 12 Jan. 2022 |
| freudenreichii | | |
| Bacillus subtilis | DSM 32324 | 8 Jun. 2016 |
| Bacillus licheniformis | DSM 17236 | 7 Apr. 2005 |

### EXAMPLES

A series of in vitro experiments was performed to evaluate the inhibitory effect of the different probiotic strains of the disclosure on Clostridium perfringens type A, type C and Salmonella Heidelberg and comparing the inhibition ability with that of other probiotic products currently available on the market. Additionally, in vivo trials have been conducted to evaluate the effects of combining different probiotic strains on health and performance of beef cattle. The strains evaluated include Lactobacillus animalis DSM33570, Propionibacterium freudenreichii DSM34127, Bacillus licheniformis DSM17236, and Bacillus subtilis DSM 32324, used at a ratio of 1.3, 1.3, 3.9, and 1.3×10⁹ colony forming units (CFU)/head per day, respectively.

### Example 1

The two pathogenic strains C. perfringens type A: DSM756 and C. perfringens type C: NCTC3180 were inoculated onto TSA-SB plate and incubated at 37° C. under anaerobic conditions overnight. The probiotic strains Lactobacillus animalis DSM33570, Bacillus licheniformis DSM17236, and Bacillus subtilis DSM 32324 were inoculated in an adequate media [brain heart infusion (BH) broth], and incubated at 37° C. aerobically for 24 h, and the probiotic strain Propionibacterium freudenreichii DSM34127 was inoculated in BHI broth, incubated at 37° C. anaerobically for 48 h. The plates for the assay were prepared as follow:
Prepare perfringens agar base in autoclave and cool to 55° C.
Make a 1.3 McFarland suspension of each C. perfringens sample.
Add 10 μL of the pathogen suspension to 35 ml perfringens agar and mix gently.
Pour the agar into omnitray plates put Immuno TSP on top immediately after.
Allow the agar to solidify for 30 min.
Remove the TSP-lids and replace with a normal monolid.
Mix the probiotic strains together as follows: 1.3×10⁹ CFU/each for Lactobacillus animalis DSM33570, Propionibacterium freudenreichii DSM34127, and Bacillus subtilis DSM 32324, and 3.9×10⁹ CFU of Bacillus licheniformis DSM17236 (Final mix pH: 6.5). Spot plates with 10 μl of the probiotic strains mix and incubateanaerobically at 42° C.

After 5 hours and 24 hours, incubation plates were evaluated for pathogen inhibition and scanned for signs of inhibition. Clear agar around the spot of probiotic strains mixture application were considered to be inhibition zones and demonstrate that the probiotic mixture of the present disclosure can inhibit the C. perfringens.

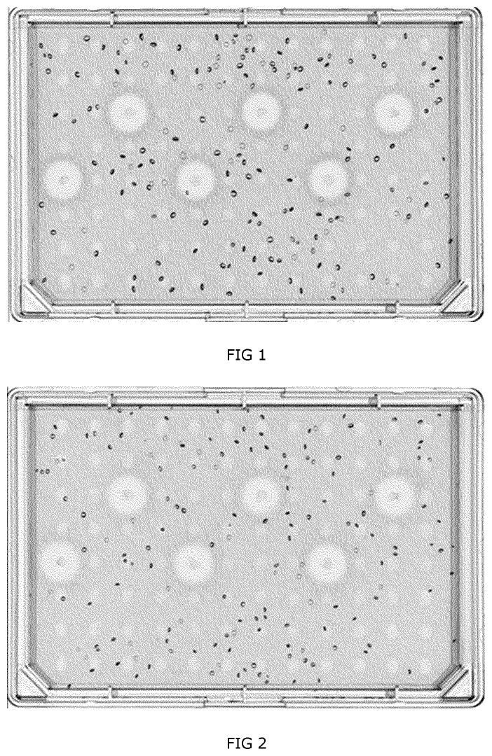

As can be seen in FIG. 1 and FIG. 2 the probiotic mixture of the disclosure (composition of the disclosure) shows an inhibitory effect against C. perfringens type A and type C (6 replicates of each). Clear zones means inhibition of the pathogen by the tested product (spotted in the wells).

### Example 2

The efficacy in inhibiting C. perfringens of the probiotic mixture of strains of the present disclosure (Lactobacillus animalis DSM33570, Propionibacterium freudenreichii DSM34127, Bacillus licheniformis DSM17236, and Bacillus subtilis DSM 32324) was evaluated and compared to the inbiting ability of different probiotics products on the market. An agar diffusion assay was performed using the same methodology as described in Example 1 to evaluate the inhibitory effects of a Bacillus-based probiotic (Bacillus subtilis; competitor #1) against C. perfringens type A, a Lactobacillus salivarius strain (competitor #2) against C. perfringens types A and C, and a lactic acid-producing strain (L. animalis; competitor #3) and/or lactic acid-utilizing strain (Acidipropionibacterium acidipropionici; competitor #4) against C. perfringens types A and C.

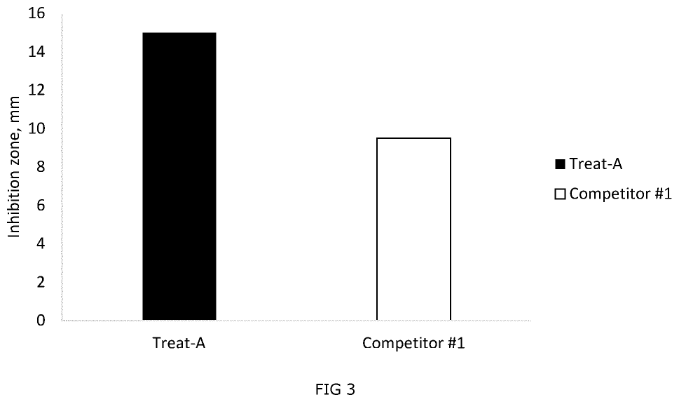

The probiotic mixture of strains of the present disclosure shows a greater inhibition zone than all of the tested competitor products (see FIGS. 3, 4 and 5).

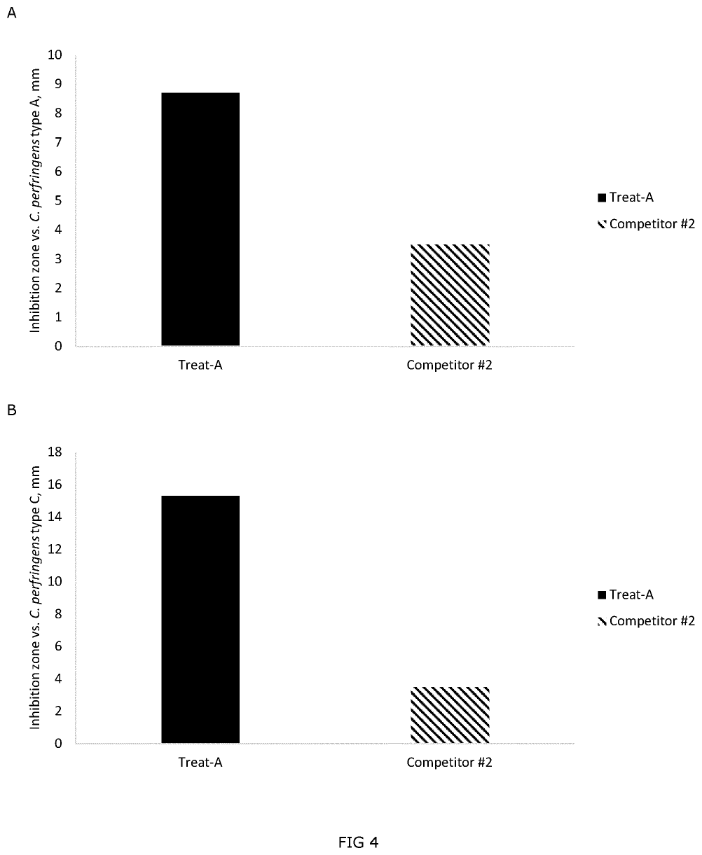

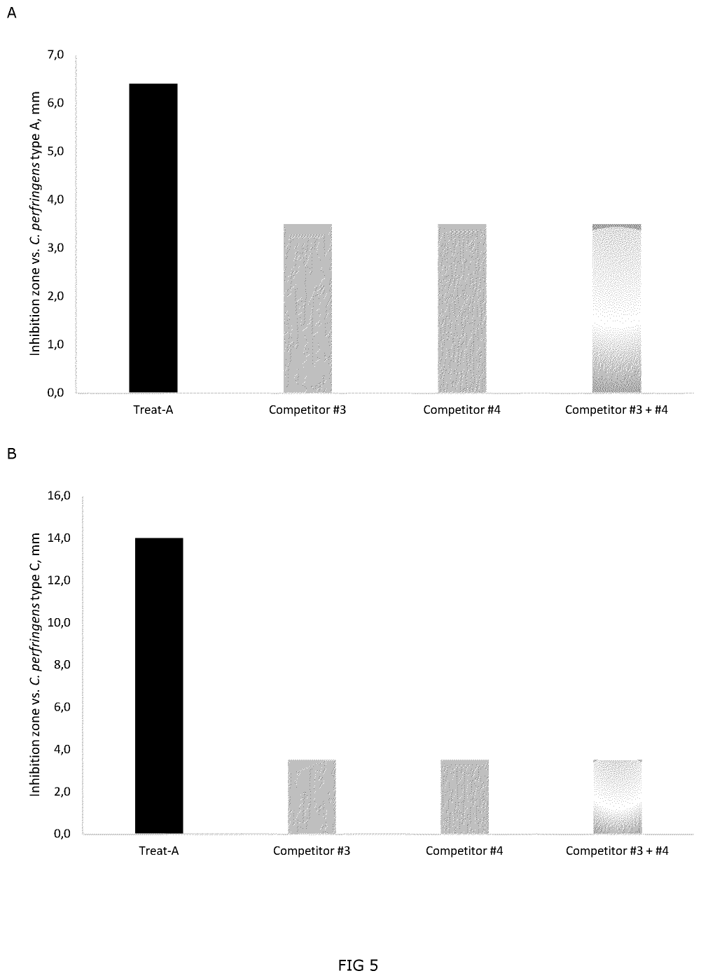

Competitor #1 shows some inhibition (FIG. 3), while competitor #2 shows a minimal/no inhibition zone (3.5 mm) against either of the C. perfringens types tested here (FIG. 4). Also for competitors #3 and #4, no inhibition was observed for any of the C. perfringens types A and C, either when tested alone or in combination (FIG. 5).

### Example 3

The efficacy of the probiotic mixture of strains of the present disclosure (Lactobacillus animalis DSM33570, Propionibacterium freudenreichii DSM34127, Bacillus licheniformis DSM17236, and Bacillus subtilis DSM 32324) to inhibit Salmonella Heidelberg in an agar diffusion assay was evaluated.

An agar diffusion assay was performed using the same methodology as described in Example 1 to evaluate the inhibitory effects of the probiotic mixture of strains of the present disclosure (Lactobacillus animalis DSM33570, Propionibacterium freudenreichii DSM34127, Bacillus licheniformis DSM17236, and Bacillus subtilis DSM 32324) this time against Salmonella Heidelberg as the target pathogen. Samples were analyzed over three runs.

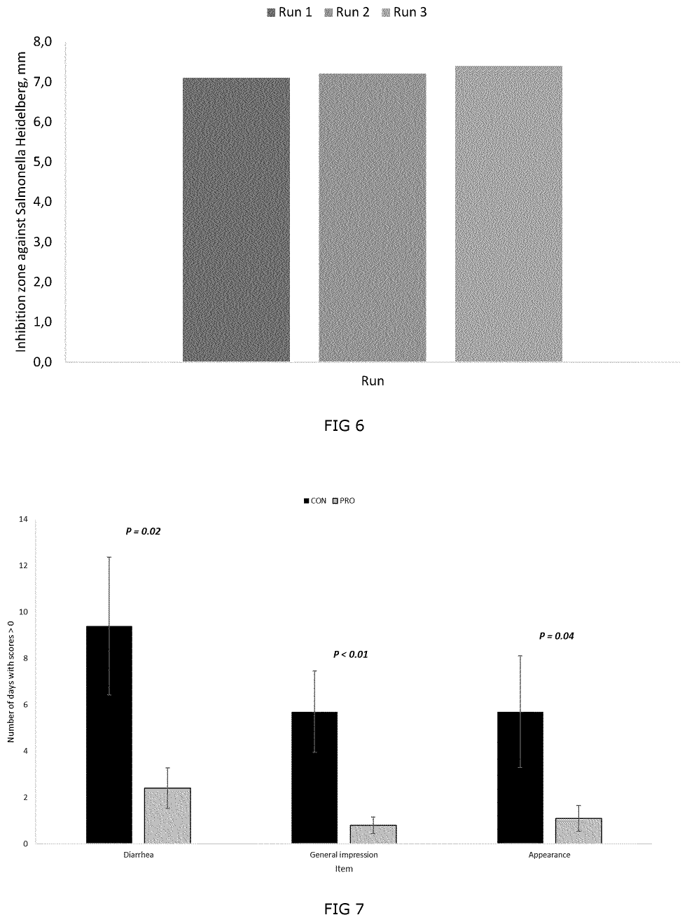

As can be seen in FIG. 6, over the three runs, the probiotic mixture of strains of the present disclosure (Treat-A) was able to inhibit Salmonella Heidelberg in the agar diffusion assay.

### Example 4

The first in vivo trial was conducted at the Midwest Veterinary Services (in vivo phase) and at the Central States Research Centre Inc. (laboratorial phase), both located in Oakland, NE, USA. In this trial, the primary work hypothesis was that the combination of four probiotic strains at different ratios would impact the health of pathogen-challenged beef calves. Therefore, the objective was to evaluate the effects of different probiotic formulations on the health of pathogen-challenged newborn beef calves.

Twenty (n=20) healthy 1-day old beef calves were assigned to one of two groups: (1) Control: no probiotic supplementation (CON; n=10), and (2) Probiotic: supplementation of the mixture comprising the present disclosure for 21 days (PRO; n=10). Seven days after the beginning of the trial, all calves were orally dosed with 1.0×10⁸ CFU of Clostridium perfringens type A strain S-107 (ATCC 13124). All animals were observed daily for health status and clinical signs of disease associated with C. perfringens infection including for health score (general impression and appearance) and diarrhea score as described herein below.

| | General | |
|---|---|---|
| Score | impression | Appearance |
| 0 | Good | Clean backside, |
| | | tail, legs |
| 1 | Mildly | Backside and tail |
| | depressed | slightly dirty |
| | | with some sticky |
| | | feces or dry |
| | | fecal material |
| 2 | Moderately | Backside and tail |
| | depressed | very dirty, not |
| | | wet, drying |
| 3 | Severely | Backside, tail, |
| | depressed, | and legs dirty |
| | difficulty rising | and wet from |
| | | watery diarrhea |
| 4* | Moribund or | |
| | dead | |

*Calf unlikely to recover, euthanasia is recommended.

| | Diarrhea Score |
|---|---|
| Score | Description |
| 0 | Normal, feces retain form, may be pasty do not flow across a smooth surface |
| 1 | Mild: form is a puddle, not a patty, sufficient water content to slowly flow down a |
| | smooth surface |
| 2 | Moderate: feces with sufficient water content to easily flow across or down a smooth |
| | surface, while leaving some adherent material |
| 3 | Severe: part or all of feces are very watery, drain away leaving little or no residue on |
| | a smooth surface (a calf may have very watery feces followed by some solid material |
| | and still have severe diarrhea). |

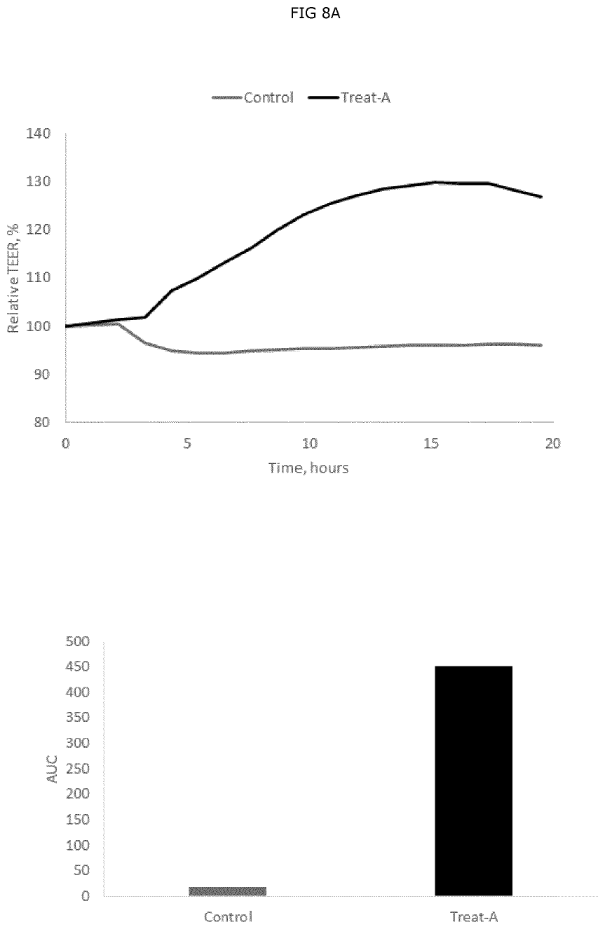

FIG. 7 reports the number of days in which animals were considered having an abnormal score, according to the aforementioned scale (General impression, Appearance and Diarrhea score). As reported, PRO supplementation (i.e., treatment with the probiotic composition of the disclosure) increased the number of days which calves were scored as “normal” (score 0; P<0.05) for diarrhea, general impression and appearance. For these results, a reduced number of days with abnormal scores (>0) would be desirable to maintain health of the animals following the encounter with a pathogen, such as C. perfringens.

Based on these results, it is feasible to conclude that:
PRO (i.e., treatment with the probiotic composition of the disclosure) reduced the number of days which animals were scored as having abnormal diarrhea and appearance score (>0) when compared with CON

### Example 5

The second in vivo trial was conducted at Texas Tech University (Lubbock, TX, USA) and aimed to evaluate the effects of the same formulations as used in Examples 1 and 2 on performance of feedlot beef cattle offered a high-concentrate diet.

In this trial, 128 crossbred beef steers (British×Continental) were used, and steers were assigned to 1 of 32 feedlot pens (4 steers/pen) according to initial body weight (BW). Pens were then assigned to one of the two groups: (1) Control: high-concentrate diet without any probiotic supplementation (CON; n=16), and (2) Probiotic: CON diet with the supplementation of the mixture comprising the composition of the disclosure (PRO; n=16). The PRO was mixed directly into the total mixed ration (TMR) that was offered to the animals throughout the experimental period (121 days) and administered to the animals in the form of a DFM. The finishing diet consisted of 65.0% steam-flaked corn, 20.0% wet corn gluten feed, 8.0% low-quality alfalfa hay, 3.0% yellow grease, 2.0% mineral-vitamin mix, 1.6% limestone, and 0.4% urea (all % dry matter basis).

Pen dry matter intake (DMI) was evaluated daily throughout the 121-day experimental period, whereas BW measurements were taken monthly, and average daily gain (ADG) was calculated in each of the 30-day periods and overall, from days 0 to 121. Feed efficiency (FE) was also calculated by dividing ADG and DMI of each pen in each 30-day period and overall, from days 0 to 121. Final BW at the feedlot (day 121) was adjusted to the carcass BW obtained at slaughter and reported as “carcass-adjusted” for all the variables analyzed in the following results presentation. At slaughter, all individual carcasses were weighed, and dressing percent (DP) was calculated by dividing carcass weight and final BW at the feedlot.

Table 3 reports the feedlot performance data from the present experiment. Treatment effects were observed on carcass-adjusted final BW (P=0.03), overall carcass-adjusted ADG (P=0.03), and overall carcass-adjusted FE (P=0.01). In all of these variables, supplementation of PRO-A (i.e., treatment with the probiotic composition of the disclosure) yielded better results on final BW (+15 kg or 2.4% improvement), ADG (+110 g/day or 6.8% improvement), and FE (+9 g ADG/kg DMI or 6.0% improvement) when compared with non-supplemented CON cohorts (P≤0.03).

TABLE 2
Feedlot performance of crossbred beef steers receiving
control supplement or probiotic supplement during a
121-day experimental period (n = 16 pens/treatment)¹

| | | | | P- |
|---|---|---|---|---|
| Item | CON | PRO | SEM | value |
| Body weight, kg | | | | |
| Initial | 409 | 408 | 7.9 | 0.69 |
| Final | 630 | 640 | 9.6 | 0.12 |
| Carcass-adjusted | 638 | 653 | 7.0 | 0.03 |
| DMI, kg/d | 10.6 | 10.6 | 0.17 | 0.85 |
| ADG, kg | | | | |
| Carcass-adjusted | 1.62 | 1.73 | 0.033 | 0.01 |
| FE, g/kg | | | | |
| Carcass-adjusted | 150 | 159 | 2.9 | <0.01 |
| Hot carcass weight, kg | 403 | 413 | 4.5 | 0.03 |

¹CON = no probiotic supplementation; PRO = supplementation of a mixture of probiotic (L. animalis, P. freudenreichii, B. licheniformis, and B. subtilis at a rate of 1.3, 1.3, 3.9, and 1.3 × 10⁹ CFU/head per day)
²SEM = standard error of the mean
³DMI = dry matter intake
⁴ADG = average daily gain
⁵FE = feed efficiency.

Following slaughter, supplementation with PRO also increased hot carcass weight (HCW; P=0.03). Hot carcass weight is obtained by weighing the edible parts of the carcass after slaughter, removing bones, internal organs, viscera etc.

Based on these results, it is feasible to conclude that:
PRO supplementation (i.e., treatment with the probiotic composition of the disclosure) increased feedlot performance of beef steers offered a high-concentrate diet, such as final BW (+15 kg or 2.4% improvement), ADG (+110 g/day or 6.8% improvement), and FE (+9 g ADG/kg DMI or 6.0% improvement) when compared with non-supplemented cohorts
PRO supplementation (i.e., treatment with the probiotic composition of the disclosure) also increased hot carcass weight by 10 kg (or 2.5% improvement)

In summary, both in vivo trials presented and discussed herein above support the following:
Formulation used for PRO (i.e., the probiotic composition of the disclosure) was able to maintain adequate health of newborn beef calves challenged with a pathogen, such as Clostridium perfringens type A.
Supplementation with the formulation used for PRO (i.e., the probiotic composition of the disclosure) yielded overall greater performance (final body weight, average daily gain, and feed efficiency) in beef steers receiving a high-concentrate diet at the feedlot
In agreement with the feedlot performance results, supplementation with the formulation used for PRO (i.e., the probiotic composition of the disclosure) resulted in heavier carcasses following slaughter.

### Example 6

The in vivo trial was initiated late Spring after one week of adaptation to the new grouping and small pens. Steers were kept on a receiving diet for 5 d after the initiation of the study and then stepped up every 5 d (4 step-up diets until steers reached the finisher diet on day 21).

Dietary treatments consisted of the following: 1) Control: no DFM (lactose-carrier only (2 g/steer [8 g/pen]) or 2) Treat-A: DFM mixture containing the probiotic mixture of strains of the present disclosure (Lactobacillus animalis DSM33570, Propionibacterium freudenreichii DSM34127, Bacillus licheniformis DSM17236, and Bacillus subtilis DSM 32324) at [1.3; 1.3; 1.3; and 3.9×10⁹ CFU/animal-daily, respectively]. All treatments were stored at −20° C. and were incorporated into the mixer daily, approximately 10 min prior to feeding.

Bunk calls were made at 1400 h every day to adjust for feed offerings if needed. Feed refusals (if present) were collected prior to feeding and weighed to adjust for daily intake. Two batches of feed were made daily using a horizontal Roto-Mix® mixer (Roto-Mix®, Kansas, USA) and offered to pens one hour after bunk calls (at 1500 h) within treatment in the following order: 1) Control and 2) Treat-A. Feed refusals were dehydrated in a forced-air oven (100° C. for 24 h) to calculate daily dry matter (DM) and subtracted from the daily amount offered of diet DM, aiming to calculate the DM intake (DMI). Additional diet and ingredient samples were collected once a week and dehydrated (55° C. for 48 h) for further diet nutrient analyses.

A digestibility assessment was conducted from days 68 to 72 of the study. During this period, refusals were collected prior to feeding, diet samples were collected from all pens during feeding and fecal samples were collected at 0700 h and 1700 h from at least three steers within each pen and frozen (−20° C.). Diet and refusal samples were dehydrated in a force-air oven (100° C. for 24 h) to calculate DMI of pens. A subsample of diets and refusals were dehydrated at 55° C. for 72 h, and ground to pass a 1 mm screen using a Wiley Mill (Thomas Scientific, Swedesboro, NJ) for further laboratorial nutrient analyses. Fecal samples were composited by pen (10 samples per pen) by using approximately 30 g (as-is) from each homogenized sample, dehydrated in a force air oven at 55° C. for 120 h, and ground to 1 mm for laboratorial nutrient analyses.

Both fecal and diet samples were analyzed for 288 h using indigestible NDF (internal marker for nutrient digestibility) according to Gregorini et al. (2008), Cole et al. (2011), and Krizsan and Huhtanen (2013), and used to estimate total fecal output and subsequent apparent total tract nutrient digestibility. Briefly, Ankom F-57 bags containing 0.5 g of diet and feces were incubated for 288 h in situ [ruminally cannulated steer offered a hay-based diet, WW B-Dahl (Bothriochloa bladhii)], rinsed, and processed with neutral detergent solution according to (Van Soest et al., 1991) with the inclusion of alpha-amylase, sodium sulfite, final acetone rinse, and without discounting residual ash. Apparent total tract digestibility was determined according to INDF, using the equation as follows: Apparent total tract nutrient digestibility was determined as follows: ATTND, %=100−100×[(conc. of INDF in feed)/(conc. of INDF in feces)×(conc. of INDF in feces)/(conc. of INDF in feed)]

Steers offered Treat-A had a greater (P=0.03) digestibility of DM (79.3 vs. 77.1%) and tended to have a greater NDF (56.9 vs. 51.6%; P=0.07), hemicellulose (59.4 vs. 53.9%; P=0.07), and ADF (47.2 vs. 52.2%; P=0.10) compared to control (Table 3).

These results highlight the ability of probiotic mixture of strains of the present disclosure to promote nutrient utilization and, therefore, the performance results (improvements on average daily gain, hot carcass weight, and feed efficiency) observed in Example 5.

TABLE 3
Apparent total tract nutrient digestibility of steers offered a steam-flaked
corn-based diet with or without supplementation of a direct-fed microbial

| Apparent | | | | |
|---|---|---|---|---|
| total | | | | |
| tract | | | | |
| nutrient | | | | |
| digestibility, % | Control | Treat-A | SEM | P= |
| Dry matter | 77.1 | 79.3 | 0.68 | 0.03 |
| Organic matter | 81.5 | 83.0 | 0.68 | 0.14 |
| NDF | 51.6 | 56.9 | 1.99 | 0.07 |
| ADF | 47.2 | 52.2 | 2.15 | 0.10 |
| Hemicellulose | 53.9 | 59.4 | 2.10 | 0.07 |

### Example 7

Six ruminally-cannulated beef steers (BW=520±30 kg) were used in a duplicated 3×3 Latin square design and offered a steam-flaked corn-based finishing diet ad libitum for three 28-d periods (21-d adaptation and 7-d collection). Treatments assigned were: 1) Control (no DFM mixture; lactose only) or 2) Treat-A: DFM mixture containing the probiotic mixture of strains of the present disclosure (Lactobacillus animalis DSM33570, Propionibacterium freudenreichii DSM34127, Bacillus licheniformis DSM17236, and Bacillus subtilis DSM 32324) at [1.3; 1.3; 1.3; and 3.9×10⁹] CFU/animal-daily, respectively. Ruminal pH and temperature were measured every 6 min with indwelling wireless probes placed in the rumen of the animals. Ruminal samples were collected at 0, 2, 4, 8, 16, and 23 h after feeding on day 28. Feed (once daily) and fecal (twice daily) samples were composited by period and analyzed. Steers offered Treat-A experienced 300 min/d less time under the ruminal pH 5.6 (P=0.04), tended to have a higher minimum pH (P=0.08), while showing a greater ruminal pH average [5.66 vs. 5.50 (P=0.05)], and had a lesser ruminal temperature [39.2 vs. 39.4° C. (P=0.02)] compared to control (Table 4).

These latter results indicate that the probiotic mixture of strains of the present disclosure supports rumen health of ruminant animals fed a high-concentrate diet, as a higher minimum pH and ruminal pH average is linked to the nutrient digestibility results observed above (Example 6).

TABLE 4
Rumen pH measurements of steers offered a steam-flaked corn-based
diet with or without supplementation of a direct-fed microbial

| Item | Control | Treat-A | SEM | P= |
|---|---|---|---|---|
| Rumen pH | | | | |
| Mean | 5.50 | 5.66 | 0.078 | 0.05 |
| Minimum | 5.09 | 5.21 | 0.117 | 0.08 |
| Maximum | 6.15 | 6.32 | 0.100 | 0.13 |
| Rumen pH < 5.6 | | | | |
| Area | 260 | 178 | 124.3 | 0.30 |
| Time, min | 928 | 627 | 220.2 | 0.04 |
| Rumen pH < 5.0 | | | | |
| Area | 8.3 | 1.3 | 5.47 | 0.35 |
| Time, min | 65 | 31 | 53.7 | 0.62 |
| Rumen temperature, ° C. | 39.4 | 39.2 | 0.19 | 0.02 |

### Example 8

Epithelial and endothelial cells form barriers in the body. The strength and integrity of these barriers can be assessed via measurements of the electrical resistance across the cell layer in vitro, called TEER. TEER is a well-established method of evaluating and monitoring epithelial tissue in a non-destructive assay. In particular, the confluence of the monolayer is quickly determined. The confluence can be tracked and monitored in real-time as the TEER measurement will rise as the gaps in the monolayer close. TEER is often used with epithelial and endothelial cells in a monolayer as a strong indicator of cell barrier integrity and permeability.

### Pro-Inflammatory Cytokine Challenge

Human cancer-derived epithelial intestinal Caco-2 cell monolayers were seeded on 1.12-cm² transwells (0.4 μm pore size, Corning) at 5×10⁴ cells/transwell per insert. Culture medium (Dulbecco's Modified Eagle Medium supplemented with non-essential amino acids, penicillin-streptomycin-amphotericin B and 10% fetal bovine serum) was changed every 3-4 days. After 20-22 days, upon reaching a confluent, polarized and differentiated state, the cells were equilibrated overnight in antibiotic-free cell culture medium in a CellZscope2 system. On the day of the experiment, the probiotic mixture of strains of the present disclosure (Treat-A) containing Lactobacillus animalis DSM33570, Propionibacterium freudenreichii DSM34127, Bacillus licheniformis DSM17236, and Bacillus subtilis DSM 32324 at a 1:1:1:3 ratio was added to the apical compartments of the cell monolayers at a concentration normalized to 1.7×10⁸ CFU per transwell. After two hours, tumor necrosis factor-α (TNF-α) and interferon-gamma (IFN-γ) were added to the basolateral compartments at a 10:1 ratio, respectively. Hourly TEER measurements were carried out for a total of 20 hours and the area under the curve (AUC) was also calculated.

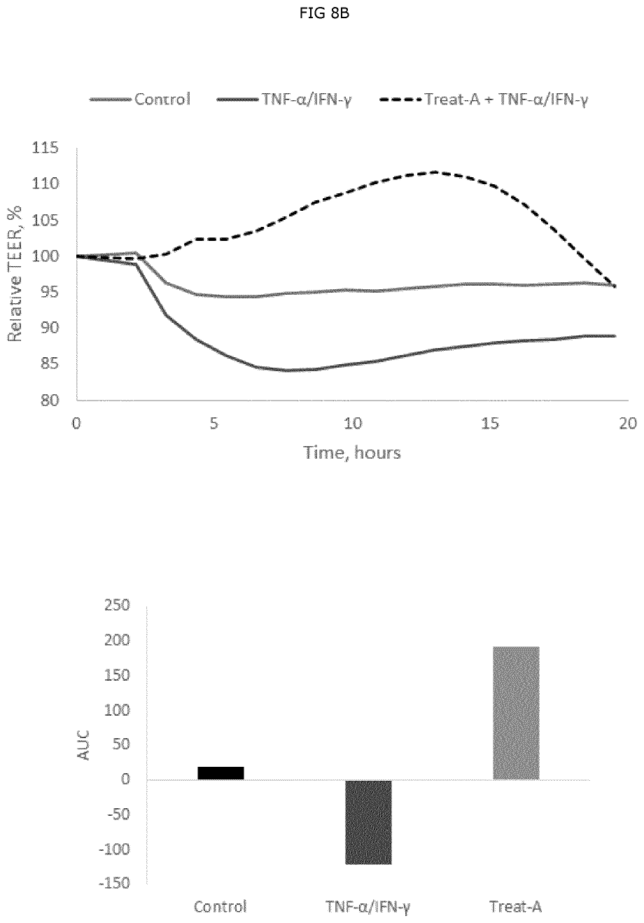

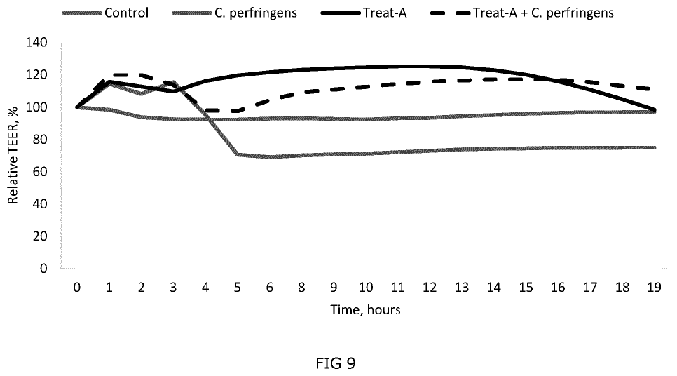

As shown in FIG. 8, exposure of Caco-2 cell monolayers to Treat-A increases TEER and AUC compared to unstimulated Caco-2 cells. When the stressor mixture (TNF-α and IFN-γ) was added basolaterally, Treat-A was able to maintain TEER and AUC over unstimulated control or negative (challenged) control.

### Pathogen Challenge

The same cells and methodology for cell culture described herein above under “Pro-inflammatory cytokine challenge” was used to test the effect on cell barrier integrity and permeability after challenge with a pathogen.

On the day of the experiment, the probiotic mixture of strains of the present disclosure (Treat-A) containing Lactobacillus animalis DSM33570, Propionibacterium freudenreichii DSM34127, Bacillus licheniformis DSM17236, and Bacillus subtilis DSM 32324 at a 1:1:1:3 ratio was added to the apical compartments of the cell monolayers at a concentration normalized to 1.0×10⁸ CFU per transwell. Clostridium perfringens type A (ATCC 13124) was added to the basolateral compartments at a dosage of 2.8×10⁷ CFU/transwell. Hourly TEER measurements were carried out for a total of 19 hours.

Exposure of Caco-2 cell monolayers to Treat-A increases TEER compared to unstimulated Caco-2 cells, but also when the pathogenic bacteria C. perfringens type A was added to the cells (FIG. 9).

In summary, in vitro administration of the probiotic mixture of strains of the present disclosure (i) supports the integrity of intestinal cells in the absence of a pathogen/stressor, (ii) supports the integrity of the intestinal barrier in the presence of pro-inflammatory cytokines (TNF/IFN) and of an important gastrointestinal pathogen (C. perfringens type A).

1. A method of inhibiting a gastrointestinal pathogenic infection in a ruminant, the method comprising, administering to said ruminant a composition that inhibits the development of clinical signs of disease associated with a pathogenic bacterium, said composition comprising probiotic strains of the follow four species: Lactobacillus animalis, Propionibacterium freudenreichii, Bacillus subtilis and Bacillus licheniformis, wherein said composition is administered to said ruminant in an amount to provide a total amount of said probiotic strains of between 1×10⁸ and 1×10¹¹ CFU/head/day.

2. The method of claim 1, further comprising assessing the effects of pathogen reduction by said administration of said composition.

3. The method of claim 1, wherein the ratio in colony forming unit (CFU) between said probiotic strains of said species is 1:1:1:3 Lactobacillus animalis:Propionibacterium freudenreichii:Bacillus subtilis:Bacillus licheniformis.

4. The method of claim 1, wherein the Lactobacillus animalis strain is the strain deposited as DSM33570.

5. The method of claim 1, wherein the Propionibacterium freudenreichii strain is the strain deposited as DSM34127.

6. The method of claim 1, wherein the Bacillus subtilis strain is the strain deposited as DSM32324.

7. The method of claim 1, wherein the Bacillus licheniformis strain is the strain deposited as DSM17236.

8. The method of claim 1, wherein the pathogenic bacterium is Clostridium perfringens.

9. The method of claim 1, wherein the pathogenic bacterium is Salmonella.

10. The method of claim 1, wherein the ruminant's number of days with diarrhea is reduced and/or the number of days the ruminant has a normal appearance is increased.

11. A method of increasing average daily weight gain of a ruminant, the method comprising:
administering to said ruminant a composition that comprises probiotic strains of the following four species Lactobacillus animalis, Propionibacterium freudenreichii, Bacillus subtilis and Bacillus licheniformis, wherein the composition is administered to said ruminant in an amount to provide a total amount of said probiotic strains of between 1×10⁸ and 1×10¹¹ CFU/day.

12. The method of claim 11, further comprising assessing the effects on average daily weight gain by said administration of said composition.

13. The method of claim 11, wherein the ratio in colony forming unit (CFU) between said probiotic strains of said species is 1:1:1:3 Lactobacillus animalis:Propionibacterium freudenreichii:Bacillus subtilis:Bacillus licheniformis.

14. The method of claim 1, wherein the Lactobacillus animalis strain is the strain deposited as DSM33570.

15. The method of claim 11, wherein the Propionibacterium freudenreichii strain is the strain deposited as DSM34127.

16. The method of claim 11, wherein the Bacillus subtilis strain is the strain deposited as DSM32324.

17. The method of claim 11, wherein the Bacillus licheniformis strain is the strain deposited as DSM17236.

18. An animal feed, animal feed additive or premix comprising a composition consisting of probiotic strains of the following four species: Lactobacillus animalis, Propionibacterium freudenreichii, Bacillus subtilis and Bacillus licheniformis, wherein said strains are present in said animal feed, animal feed additive or premix in an amount to provide a total of between 1×10⁸ to 1×10¹¹ CFU/head/day of said probiotic strains, wherein said animal feed, animal feed additive or premix further comprises one or more additional components selected from concentrate(s), vitamin(s), mineral(s), enzyme(s), amino acid(s), and other feed ingredient(s).
